(19) United States
(12) Patent Application Publication
(10) Pub. No.: US 2009/0297484 A1
(43) Pub. Date: Dec. 3, 2009

(54) METHOD FOR CURING AND PREVENTING ACQUIRED IMMUNE DEFICIENCY SYNDROME BY ALTERING CELL-SURFACE RECEPTORS IN PRECURSOR T-HELPER CELLS AND MATURE T-HELPER CELLS TO PREVENT HUMAN IMMUNODEFICIENCY VIRUS VIRIONS ACCESS TO MATURE HELPER CELLS

(76) Inventors: Lane Bernard Scheiber, Annandale, VA (US); Lane Bernard Scheiber, II, Grosse Ile, MI (US)

Correspondence Address: LANE B. SCHEIBER, 7864 OAK RIVER DRIVE, GROSS ILE, MI, 48138, US

(21) Appl. No.: 12/131,149

(22) Filed: Jun. 2, 2008

(51) Int. Cl. A61K 48/00; A61P 31/18

(52) U.S. Cl. 424/93.6

(57) ABSTRACT

The medical method by which a modified Human Immunodeficiency Virus or virus-like structure is used as a transport medium to carry a medically therapeutic ribonucleic acid to precursor T-Helper cells and mature T-Helper cells to prevent AIDS. The modified Human Immunodeficiency Virus or virus-like structures make contact with precursor T-Helper cells or mature T-Helper cells by means exterior probes. Once the exterior probes engage the precursor T-Helper cells' or mature T-Helper cells' cell-surface receptors, the modified virus or virus-like structures inserts into the cells the medically therapeutic ribonucleic acid they are carrying. The medically therapeutic ribonucleic acid causes T-Helper cells to express an altered cell-surface receptor on their surface thus thwarting HIV virions from being able to gain access to such cells, thus preventing AIDS.

### CROSS-REFERENCE TO RELATED APPLICATIONS

None.

### STATEMENT REGARDING SPONSORED RESEARCH OR DEVELOPEMNT

Not applicable.

### REFERENCE TO SEQUENCE LISTING, A TABLE, OR COMPUTER LISTING COMPACT DISC APPENDIX

Not applicable.
© Lane B. Scheiber and Lane B. Scheiber II. A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device that is intended to alter the cell-surface receptors on precursor T-Helper cells or mature T-Helper cells in order to make an individual immune to an infection by the Human Immunodeficiency Virus.

2. Description of Background Art

It has been estimated by the Center for Disease Control that in the United States 55,000 to 60,000 new cases of Human Immunodeficiency Virus (HIV) are occurring each year. It is thought that there are 900,000 people currently infected with HIV in the United States, with many victims not aware that they have contracted the virus. Further, it has been estimated that the Human Immunodeficiency Virus (HIV), the pathogen that causes Acquired Immune Deficiency Syndrome (AIDS), has infected as many as 30-60 million people around the globe.

The presence of HIV was first came to the attention of those in the United States in 1981, when there appeared an outbreak of Kaposi's Sarcoma and Pneumocystis carinii pneumonia in gay men in New York and California. After over twenty-five years of research and investigation, eradicating the ever growing global humanitarian crisis posed by the HIV remains an elusive goal for the medical community. It is estimated the virus has already killed 25 million citizens of this planet.

The Human Immunodeficiency Virus has been previously referred to as human T-Lymphotrophic virus III (HTLV-III), lymphadenopathy-associated virus (LAV), and AIDS-associated retrovirus (ARV). Infection with HIV may occur by the virus being transferred by blood, semen, vaginal fluid, or breast milk. Four major means of transmission of HIV include unprotected sexual intercourse, contaminated needles, breast milk, and transmission from an infected mother to her baby at birth.

HIV is an ingeniously constructed very deadly virus, which represents the most challenging pathogen the worldwide medical community faces to date. A pathogen may be a bacterium, virus, or other organism that may cause disease. Viruses in general, have been difficult to contain and eradicate due to the fact they are obligate parasites and tend not to carry out any biologic functions outside the cell the virus has targeted as its host. A virus when it exists outside the boundaries of a cell is generally referred to as a virion. HIV virions posses several attributes that make them very elusive and difficult to destroy.

For purposes of this text, the term ‘body’ refers to the material part of a man or a woman, generally including the head, neck, trunk, extremities and all usual internal structures. The terms ‘body’ and ‘human body’ are interchangeable.

Bacterial infections have posed an easier target for the medical community to eradicate from the body. Bacteria generally live and reproduce outside animal cells. Bacteria, like animal cells, carryout biologic functions. A large multi-celled organism such as the human body combats bacterial infections with a combined force of white cells, antibodies, complements and its lymphatic system. White cells circulate the body in search of bacteria. When a white cell encounters a bacterium, the white cell engulfs the bacterium, encapsulates the pathogen, processes the identification of the pathogen and kills the pathogen utilizing acids and destructive enzymes. The white cell then alerts the B-cells of the immune system as to the identity of the intruding bacterium. A subpopulation of B-cells is generated, dedicated to producing antibodies directed against the particular pathogen the circulating white cell encountered and identified. Antibodies, generated by B-cells, traverse the blood and body tissues in search of the bacteria they were designed to repel. Once an antibody encounters a bacterium it is targeted to attack, the antibody attaches to the bacterium's outer wall. The effect antibodies have in coating the outside of a bacterium is to assist the white cells and the other components of the immune system in recognizing the bacterium, so that appropriate defensive action can be taken against the pathogen. Some antibodies, in addition to coating the bacterium, will act to punch holes through the bacterium's outer wall. If the integrity of the bacterium's cell wall is breached, this action generally leads to the death of the bacterium. Complements are primitive protein structures that circulate the blood stream in search of anything that appears consistent with a bacteria cell wall. Complements are indiscriminant. Once the complement proteins locate any form of bacterial cell wall, the complement proteins organize, and much like antibodies, act in concert to punch one or more holes though a bacterium's cell wall to compromise the viability of the bacterium. The lymphatic system is a diffuse network of thin walled vessels that drain excess water from extracellular fluids and join to form the thoracic duct and right lymph duct, which empty into the venous system near the heart. Lymph nodes are present at different locations in the body and screen the fluid transiting the lymphatic system, called lymph, to remove pathogens. Cells in the spleen screen the blood in search of bacteria. When a bacterial pathogen is identified, such as by antibodies coating the surface, the bacterium is taken out of circulation and terminated.

Viruses pose a much different infectious vector to the body's defense system than either bacteria or cellular parasites. Since viruses do not carry out biologic processes outside their host cell, a virus can be destroyed, but they cannot be killed. A virus is simply comprised of one or more external shells and a portion of genetic material. The virus's genetic information is carried in the core of the virus. Antibodies can coat the exterior of a virus to make it easier for the white cells in the body to identify the viral pathogen, but the action of punching holes in the virus's external shell by antibodies or complement proteins does not necessarily kill the virus. Viruses also only briefly circulate in the blood and tissues of the body as an exposed entity. Using exterior probes, a virus hunts down a cell in the body that will act as an appropriate host so that the virus can replicate. Once the virus has found a proper host cell, the virus inserts its genome into the host cell. To complete its life-cycle, the virus's genetic material takes command of cellular functions and directs the host cell to make replicas of the virus.

Once the virus's genome has entered a host cell, the virus is in effect shielded from the body's immune system defense mechanisms. Inside a host cell, the presence of the virus is generally only represented as genetic information incorporated into the host cell's DNA. Once a virus has infected a cell in the body, the presence of the virus can only be eradicated if the host cell is destroyed. Antibodies and complements are generally designed not to attack the autologous tissues of the body. Circulating white cells and the immune cells which comprise lymph nodes and the spleen may or may not recognize that a cell, which has become a host for a virus, is infected with a virus's genome. If the immune system fails to identify a cell that has become infected with a virus, the virus's genetic material can proceed to force the infected cell to make copies of the virus. Since a virus is in essence simply a segment of genetic material, time is of no consequence to the life-cycle of the virus and a virus's genome may be carried for years by the host without a need to activate; such viruses are often termed latent viruses. A virus's genetic material may sit idle in a host cell for an extended period of time until the pathogen's programming senses the time is right to initiate the virus's replication process or an action of the host cell triggers the virus to replicate. The only opportunity for the immune system to destroy a latent virus is when copies of the virus leave the host cell and circulate in the blood or tissues in search of another perspective host cell.

The traditional medical approach to combating infectious agents such as bacteria and cellular parasites, therefore has limited value in managing or eradicating elusive or latent viral infections. Synthetic antibiotics, generally used to augment the body's capacity to produce naturally occurring antibodies against bacterial infections, have little success in combating latent viral infections. Stimulating the body's immune system's recognition of a virus by administering a vaccine also has had limited success in combating elusive viral infections. Vaccines generally are intended to introduce to the body pieces of a bacteria or virus, or an attenuated, noninfectious intact bacteria or virus so that the immune system is able to recognize and process the infectious agent and generate antibodies directed to assist in killing the pathogen. Once the immune system has been primed to recognize an intruder, antibodies will be produced by the immune system in great quantities in an effort to repel an invader. Over time, as the immune system down-regulates its antibody production in response to a lack of detecting the presence of the intruding pathogen, the quantity of antibodies circulating in the blood stream may decrease in number to a quantity that is insufficient to combat a pathogen. Since antibodies have limited value in combating some of the more elusive viruses that hibernate in host cells, vaccines have limited value in destroying latent viruses.

The Human Immunodeficiency Virus demonstrates four factors which make this pathogen particularly elusive and a difficult infectious agent to eradicate from the body. First: the host for HIV is the T-Helper cell. The T-Helper cell is a key element in the immune system's response since it helps coordinate the body's defensive actions against pathogens seeking to invade the body's tissues. In cases of a bacterial infection versus a viral infection, T-Helper cells actively direct which immune cells will rev-up in response to the infectious agent and engage the particular pathogen. Since HIV infects and disrupts T-Helper cells, coordination of the immune response against the virus is disrupted, thus limiting the body's capacity to mount a proper response against the presence of the virus and produce a sufficient action to successfully eradicate the virus.

Second: again, latent viruses such as HIV, have a strategic advantage. When the immune system first recognizes a pathogen and begins to generate antibodies against a particular pathogen, the response is generally robust. Once time has passed and the immune system fails to detect an active threat, the production of antibodies against the particular pathogen diminishes. When HIV infects a T-Helper cell, the viral genome may lay dormant, sometimes for years before taking command of the T-Helper cell's biologic functions. HIV may, therefore, generate a very active initial immune response to its presence, but if the virus sits dormant inside T-Helper cells for months or years, the antibody response to the virus will diminish over time. There may not be an adequate quantity of circulating antibodies to actively engage the HIV virions as they migrate from the T-Helper cell that generated the copies to uninfected T-Helper cells that will serve as a new host to support further replication. If the immune system's response is insufficient during the period while the virus is exposed and vulnerable, it becomes extremely difficult for the body to eradicate the virus.

Third, when replicas of the Human Immunodeficiency Virus are released from their host cell, during the budding process the HIV virion coats itself with an exterior envelope comprised of a portion of the plasma membrane from the T-Helper cell that acted as the host for the virus. A T-Helper cell's plasma membrane is comprised of a lipid bilayer, a double layer of lipid molecules oriented with their polar ends at the outside of the membrane and the nonpolar ends in the membrane interior. The virus thus, in part, takes on an external appearance of a naturally occurring cell in the body. Since the exterior envelope of a HIV virion has the characteristics of a T-Helper cell it is more difficult for the immune system to recognize that it is a pathogen as it migrates through the body in search of another T-Helper cell to infect.

Fourth, the Human Immunodeficiency Virus exhibits a very elusive mode of action which the virus readily utilizes to actively defeat the body's immune system. HIV carries in its genome a segment of genetic material that directs an infected T-Helper cell to create and mount on the surface the plasma membrane a FasL cell-surface receptor. Healthy T-Helper cells carry on the surface of their plasma membrane Fas cell-surface receptors. The Fas cell-surface receptor when engaged by a FasL cell-surface receptor on another cell, initiates apoptosis in the cell carrying the Fas cell-surface receptor. Apoptosis is a biologic process that causes a cell to terminate itself. A T-Helper cell infected with the HIV virus carrying a FasL cell-surface receptor is therefore capable of killing noninfected T-Helper cells that the infected T-Helper cell encounters as it circulates the body. The occurrence of AIDS is therefore propagated not only by the number of T-Helper cells that become incapacitated due to direct infection by HIV, but also by the number of noninfected T-Helper cells that are eliminated by coming in direct contact with infected T-Helper cells.

Acquired Immune Deficiency Syndrome (AIDS) occurs as a result of the number of circulating T-Helper cells declining to a point where the immune system's capacity to mount a successful response against opportunistic infectious agents is significantly compromised. The number of viable T-Helper cells declines either because they become infected with the HIV virus or because they have been killed by encountering a T-Helper cell infected with HIV. When there is an insufficient population of non-HIV infected T-Helper cells to properly combat infectious agents such as Pneumocystis carinii or cytomegalo virus or other pathogens, the body becomes overwhelmed with the opportunistic infection and the patient becomes clinically ill. In cases where the combination of the patient's compromised immune system and medical assistance in terms of synthetic antibiotics intended to combat the opportunistic pathogens, fluids, intravenous nutrition and other treatments are not sufficient to sustain life, the body succumbs to the opportunistic infection and death ensues.

The Human Immunodeficiency Virus locates its host by utilizing probes located on its envelope. The HIV virion has two types of glycoprotein (gp) probes attached to the outer surface of its exterior envelope. A glycoprotein is a structure comprised of a protein component and a lipid component. HIV utilizes a glycoprotein 120 (gp 120) probe to locate a Cluster Designation (CD) 4 cell-surface receptor on the plasma membrane of a T-Helper cell. The plasma membrane of the T-Helper cell is comprised of a lipid bilayer. Cell-surface receptors are anchored in the lipid bilayer. Once an HIV gp 120 probe has successfully engaged a CD4 cell-surface receptor on a T-Helper cell a conformational change occurs in the gp 120 probe and a glycoprotein 41 (gp 41) probe is exposed. The gp 41 probe's intent is to engage a CXCR4 or CCR5 cell-surface receptor on the plasma membrane of the same T-Helper cell. Once a gp 41 probe on the HIV virion engages a CXCR4 or CCR5 cell-surface receptor, the HIV virion opens an access portal through the T-Helper cell's plasma membrane.

Once the virus has gained access to the T-Helper cell by opening a portal through the cell's outer membrane the virion inserts two positive strand RNA molecules approximately 9500 nucleotides in length. Inserted along with the RNA strands are the enzymes reverse transcriptase, protease and integrase. Once the virus's genome gains access to the interior of the T-Helper cell, in the cytoplasm the pair of RNA molecules are transformed to deoxyribonucleic acid by the reverse transcriptase enzyme. Following modification of the virus's genome to DNA, the virus's genetic information migrates to the host cell's nucleus. In the nucleus, with the assistance of the integrase protein, the virus's DNA becomes inserted into the T-Helper cell's native DNA. When the timing is appropriate, the now integrated viral DNA, becomes read by the host cell's polymerase molecules and the virus's genetic information commands certain cell functions to carry out the replication process to construct copies of the human deficiency virus.

Present anti-viral therapy has been designed to target the enzymes that assist the HIV genome with the replication process. Anti-viral therapy is intended to interfere with the action of these replication enzymes. Part of the challenge of eradicating HIV is that once the virus inserts its genome into a T-Helper cell host, the viral genome may lay dormant until the proper circumstances evolve. The virus's genome may sit idle inside a T-Helper cell for years before becoming activated, causing drugs that interfere with HIV's life cycle to have limited effect on eliminating the virus from the body. Arresting the replication process does not insure that T-Helper cells infected with HIV do not continue to circulate the body killing noninfected T-Helper cells thus causing the patient to progress to a clinically apparent state of Acquired Immune Deficiency Syndrome and eventually succumbing to an opportunistic infection which eventually results in the death of the individual.

The outer layer of the HIV virion is comprised of a portion of the T-Helper cell's outer cell membrane. In the final stage of the replication process, as a copy of the HIV capsid, carrying the HIV genome, buds through the host cell's plasma membrane, the capsid acquires as its outermost shell a wrapping of lipid bilayer from the host cell's plasma membrane. Vaccines are generally comprised of pieces of a virus or bacterium, or copies of the entire virus or bacterium weakened to the point the pathogen is incapable of causing an infection. These pieces of a pathogen or copies of a nonvirulent pathogen prime the immune system such that a vaccine intent is to cause B-cells to produce antibodies that are programmed to seek out the surface characteristics of the pathogen comprising the vaccine. In the case of HIV, since the surface of the pathogen is an envelope comprised of lipid bilayer taken from the host T-Helper cell's plasma membrane, a vaccine comprised of portions of the exterior envelope of the HIV virions might not only target HIV virions, but might also have deleterious effects on the T-Helper cell population. Some antibodies produced to combat HIV infections may not be able to tell the difference between an HIV virion and a T-Helper cell, and such antibodies may act to coat and assist in the elimination of both targets. In such a scenario, since such a vaccine might cause a decline in the number of available T-Helper cells, it is conceivable that a vaccine comprised of portions of the external envelope of HIV virions might paradoxically induce clinically apparent AIDS in a patient that a vaccine has been administered.

It is clear that the traditional approach of utilizing antibiotics or providing vaccines to stimulate the immune system to produce endogenous antibodies, by themselves, is an ineffective strategy to manage a virus as elusive and deadly as HIV. Drugs that interfere with the replication process of HIV generally slow progression of the infection by the virus, but do not necessarily eliminate the virus from the body nor eliminate the threat of the clinical symptoms of AIDS. A new strategy is required in order to successfully combat the threat of HIV.

A eukaryote refers to a nucleated cell. Eukaryotes comprise nearly all animal and plant cells. A human eukaryote or nucleated cell is comprised of an exterior lipid bilayer plasma membrane, cytoplasm, a nucleus, and organelles. The exterior plasma membrane defines the perimeter of the cell, regulates the flow of nutrients, water and regulating molecules in and out of the cell, and has embedded into its structure cell-surface receptors that the cell uses to detect properties of the environment surrounding the cell membrane. Cytoplasm refers to the entire contents inside the cell except for the nucleus and acts as a filling medium inside the boundaries of the plasma cell membrane. Cytosol refers to the semifluid portion of the cytoplasm minus the mitochondria and the endoplasmic reticulum. The nucleus, organelles, and ribosomes are suspended in the cytosol. Nutrients such as amino acids, oxygen and glucose are present in the cytosol. The nucleus contains the majority of the cell's genetic information in the form of double stranded deoxyfibonucleic acid (DNA). Organelles generally carry out specialized functions for the cell and include such structures as the mitochondria, the endoplasmic reticulum, storage vacuoles, lysosomes and Golgi complex. Floating in the cytoplasm, but also located in the endoplasmic reticulum and mitochondria are ribosomes. Ribosomes are protein structures comprised of several strands of proteins that combine and couple to a messenger ribonucleic acid (mRNA) molecule. More than one ribosome may be attached to a single mRNA at a time. Ribosomes decode genetic information coded in a mRNA molecule and manufacture proteins to the specifications of the instruction code physically present in the mRNA molecule.

The majority of the deoxyribonucleic acid (DNA) in a cell is present in the form of chromosomes, the double stranded helical structures located in the nucleus of the cell. DNA in a circular form, can also be found in the mitochondria, the powerhouse of the cell, an organelle that assists in converting glucose into usable energy molecules. DNA represents the genetic information a cell needs to manufacture the materials it requires to develop to its mature form, sustain life and to replicate. Genetic information is stored in the DNA by arrangements of four nucleotides referred to as: adenine, thymine, guanine and cytosine. DNA represents instruction coding, that in the process known as transcription, the DNA's genetic information is decoded by transcription protein complexes referred to as polymerases, to produce ribonucleic acid (RNA). RNA is a single strand of genetic information comprised of coded arrangements of four nucleotides: adenine, uracil, guanine and cytosine. The physical difference in the construction of a DNA molecule versus a RNA molecule is that DNA utilizes the nucleotide ‘thymine’, while RNA molecules utilize the nucleotide ‘uracil’. RNAs are generally classified as messenger RNAs (mRNA), transport RNAs (tRNA) and ribosomal RNAs (rRNA).

The nucleic acids comprising the DNA are arranged in a biologic code format. Adenine is ‘A’; Thymine is ‘T’; Cytosine is ‘C’; Guanine is ‘G’. It is believed that the DNA is divided into segments of three nucleic acids. A segment of three nucleic acids is referred to as a codon. Given there are four different nucleic acids and three nucleic acids are used to comprise a codon, there are 64 possible combinations. Most of the combinations are thought to code for amino acids, the building blocks of proteins. There are 20 amino acids. As an example, the three nucleic acid codon ‘TCA’ codes for the amino acid Serine. As another example, the three nucleic acid codon ‘GCA’ codes for the amino acid Alanine. Three of the codons are considered STOP codes. STOP codons are biologic signals to terminate events, such as reading and deciphering of the genetic code. The three STOP codes are represented in the DNA by the codons: ‘TAA’, ‘TAG’, and ‘TGA’. Since RNA is comprised of coded arrangements of the four nucleotides: adenine, uracil, guanine and cytosine, the three STOP codes are represented in the messenger RNA as the three codons: ‘UAA’, ‘UAG’ and ‘UAG’.

Proteins are generated when ribosomes decipher the genetic code present on a strand of messenger RNA. Each codon represents an amino acid. Some of the twenty amino acids have more than one codon that codes for the same amino acid. Ribosomes build proteins by stringing together amino acids as dictated by the codons present on messenger RNA. When a protein is being generated by ribosomes, the ribosomes cease reading the messenger RNA when they encounter any of the three STOP codons; the presence of a STOP code terminates production of the polypeptide chain by the ribosomes. The polypeptide chain is then released from the ribosome and represents an individual protein.

Proteins are comprised of a series of amino acids bonded together in a linear strand, sometimes referred to as a chain; a protein may be further modified to be a structure comprised of one or more similar or differing strands of amino acids bonded together. A protein comprised of one or more strands of amino acids (referred to as subunits) may be referred to as a protein complex. Insulin is a protein structure comprised of two strands of amino acids, one strand comprised of 21 amino acids long and the second strand comprised of 30 amino acids; the two strands attached by two disulfide bridges. There are an estimated 30,000 different proteins the cells of the human body may manufacture. The human body is comprised of a wide variety of cells, many with specialized functions requiring unique combinations of proteins and protein structures such as glycoproteins (a protein combined with a carbohydrate) to accomplish the required task or tasks a specialized cell is designed to perform. Forms of glycoproteins are known to be utilized as cell-surface receptors.

Viruses are obligate parasites. Viruses simply represent a carrier of genetic material and by themselves viruses are unable to replicate or carry on any form of biologic function outside their host cell. Viruses are generally comprised of one or more shells constructed of one or more layers of protein or lipid material, and inside the outer shell or shells, a virus carries a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's probes is to locate and engage a host cell's receptors. The virus's surface probes are designed to detect, make contact with and functionally engage one or more receptors located on the exterior of a cell type that will offer the virus the proper environment in which to construct copies of itself. A host cell is a cell that provides the virus the proper biochemical machinery for the virus to successfully replicate itself.

Protected by the outer coat generally comprised of an envelope or capsid or envelope and capsid, viruses carry a genetic payload in the form of deoxyribonucleic acid (DNA) or ribonucleic acid (RNA). Once a virus's exterior probes locate and functionally engage the surface receptor or receptors on a host cell, the virus inserts its genetic payload into the interior of the host cell. In the event a virus is carrying a DNA payload, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate their RNA being converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the host cell's nuclear DNA and simply utilize portions of its innate viral genome to act as messenger RNA (mRNA). RNA viruses that bypass the host cell's DNA, cause the cell, in general, to generate copies of the necessary parts of the virus directly from the virus's RNA genome. When a virus's genome directly acts as a template, then similar to the cell's messenger RNA, the virus's RNA is read by the cell's ribosomes and proteins necessary to complete the virus's replication process are generated.

The Plague or Black Death ravaged Europe between 1347 to 1350 and claimed nearly a quarter of all who lived in Europe at the time. The bubonic plague was caused by Yersinia pestis, a bacterium that lived inside rats and could be spread to humans by the bite of a flea. Yersinia binds to white blood cells and injects a toxin into the white blood cell to stun the immune system, allowing Yersinia to flourish without being attacked by the immune system. The belief is that Yersinia uses the CCR5 cell-surface receptor on white blood cells to target white blood cell and bind to them. Those who were born without the gene for CCR5 or carried a mutant gene for the CCR5 cell-surface receptor were protected from the Black Death. Today, some of the descendants of the survivors of the Black Plaque are resistant to becoming infected by HIV.

Once the 120 gp probe on an HIV virion engages a CD4 cell-surface receptor on a T-Helper cell, to gain access and infect the T-Helper cell the HIV virion's gp 41 probe must successfully engage a CXCR4 or CCR5 cell-surface receptor on the T-Helper cell. If a HIV virion's gp 41 probe is unable to properly engage a CXCR4 or CCR5 cell-surface receptor an access portal will not open up on the surface of the T-Helper cell and the cell membrane will remain impenetrable. At least one mutant CCR5 gene has been identified in the population. The mutation to the CCR5 gene is in the form of a missing 32-base section of the gene. Those who carry two copies of the mutant CCR5 gene have no CCR5 receptors on the surface of their cells and have a significantly lower rate of infection with HIV than those that carry normal CCR5 genes. Those who carry one mutant CCR5 gene have fewer CCR5 cell-surface receptors on the surface of their cells and though they may become infected with HIV, the mutation retards the onset of clinically apparent AIDS.

T-Helper cells undergo a complex maturation process to become a mature T-Helper cell. Precursor T-Helper cells are originally derived from stem cells located in the bone marrow. As precursor T-Helper cells progress through the maturation process to become a mature T-Helper cell cell-surface receptors (sometimes referred to as markers) change. Precursor T-Helper cells lack certain cell-surface receptors found on mature T-Helper cells. Stem cells present in the bone marrow may express CD 7 and CD34 cell-surface markers that are not present on mature T-Helper cells. Precursor T-Helper cells released from the bone marrow are generally do not carry CD4 or CD8 cell-surface receptors and are often termed ‘double negative’ cells, meaning they do not express either one of these cell surface receptors on their surface. Precursor T-Helper cells sometimes referred to as precursor T-Lymphocytes migrate from the bone marrow to thymus to undergo further maturation. The thymus is a gland in the neck which cultivates immature T-Helper cells through various stages of development until a mature form of T-Helper cell is reached and released into circulation. As a result of the stages of the maturation process precursor T-Helper cells continue to change some of the cell-surface receptors expressed on their surface. At one point in the maturation process, precursor T-Helper cells will express both CD4 and CD8 cell-surface receptors and be termed as double positive cells. As the precursor T-Helper cell continues to mature it will lose the expression of either the CD8 cell-surface receptors. Mature T-Helper cells express CD4 cell-surface receptors on their surface, but do not express CD 8 cell-surface receptors. Other cell-surface receptors that are expressed include CD25, CD38 and CD44.

Current state of gene therapy generally refers to efforts directed toward inserting an exogenous subunit of DNA, that are ‘known genes’ as they occur in nature, into a vehicle such as a naturally occurring virus. The vehicle is intended to insert the exogenous subunit of DNA into a target cell. The exogenous DNA subunit then migrates to the target cell's nucleus. The exogenous DNA subunit then inserts into the native DNA of the cell. This represents a permanent alteration of the cell's nuclear DNA. At some point, the nuclear transcription proteins read the exogenous DNA subunit's nucleotide coding to produce the intended cellular response.

An approach to defeating HIV from infecting healthy mature T-Helper cells would be to insert into precursor T-Helper cells a means to alter the genes that code for the CXCR4 cell-surface receptor or the CCR5 cell-surface receptor, thus producing mature T-Helper cells that do not express these cell-surface receptors or express altered, biologically nonfunctional forms of these cell-surface receptors. By generating T-Helper cells that do not express CXCR4 or CCR5 cell-surface receptors, or by generating T-Helper cells that do not express biologically functional CXCR4 or CCR5 cell-surface receptors makes these T-Helper cells impervious to infection by Human Immunodeficiency Virus virions. The natural selection process caused by the Yersinia pestis demonstrates that a substantial number of people possess mutant genes which cause T-Helper cells not express CCR5 cell-surface receptors and apparently such individuals live normal lives; thus such an alteration to cell-surface receptors does not produce a readily apparent harmful effect.

Since during the maturation process a precursor T-Helper cell expresses a CD8 cell-surface receptor and then removes this CD8 cell-surface receptor from its surface; there exits biologic instruction code and a mechanism inside the cell to remove a cell-surface receptor from the surface of a T-Helper cell or alter the cell-surface receptor such that it becomes biologically nonfunctional. Utilizing this natural process of removing or altering cell-surface receptors already present in cells, leads to removing or altering cell-surface receptors present on existing cell to make such cell impervious to infection. Removal of CXCR4 cell-surface receptors and CCR5 cell-surface receptors from the surface of mature T-Helper cells or making CXCR4 cell-surface receptors and CCR5 cell-surface receptors nonfunctional would make mature T-Helper cells impervious to infection by either Human Immunodeficiency Virus virions and Yersinia pestis and possibly other infectious pathogens.

Utilizing the natural process that a cell possesses to remove or alter cell-surface markers that exist on the surface of the cell, could lead to causing T-Helper cells infected with the Human Immunodeficiency Virus genome that are expressing FasL cell-surface receptors that terminate healthy T-Helper cells to remove their FasL receptor or alter the FasL receptor such that the FasL receptor becomes biologically nonfunctional. Removing the threat of infected T-Helper cells utilizing their FasL cell-surface receptors from terminating healthy T-Helper cells would help avert a person infected with HIV from progressing to AIDS.

The method described within the scope of this text involves utilizing modified virus virions to deliver to specific target cells such as stem cells, precursor T-Helper cells and mature T-Helper cells a medically therapeutic RNA genome as a payload. As with HIV, the RNA genome would be converted to DNA by enzymes and then inserted into the native DNA of the target cell to produce a biologic response. The biologic response would include causing a cell-surface receptor not to be expressed on the surface of the cell, or causing an altered form of a cell-surface receptor to be expressed on the surface of the cell, or causing removal of a cell-surface receptor from the surface of the cell, or causing alteration to an existing cell-surface receptor being expressed on the surface of the cell to produce a medically therapeutic outcome.

A means of treating AIDS could be to take the naturally occurring Human Immunodeficiency Virus (HIV) which is an RNA virus and replace HIV's innate genome with a genome that codes for either prevention of the CXCR4 or CCR5 cell-surface receptors from being expressed, or expression of nonfunctional CXCR4 or CCR5 cell-surface receptors or removal of existing CXCR4 or CCR5 cell-surface receptors. HIV already posses the means to locate and infect T-Helper cells, which act as the natural host for HIV replication. Modified HIV virions could be utilized to carry their modified genome to either precursor T-Helper cells or mature T-Helper cells. Utilizing modified HIV virions to insert modified genome in a patient's T-Helper cells would prevent HIV by altering the cell-surface receptors of the patient's T-Helper cell population, preventing HIV from being able to access T-Helper cells and thus stop AIDS from occurring or progressing in a patient.

### BRIEF SUMMARY OF THE INVENTION

The medical treatment device comprised of a modified virus or virus-like structure is used as a transport medium to carry a payload consisting of a modified genome that alters the CXCR4 or CCR5 cell-surface receptors present on the surface of the T-Helper cells in the body. The modified virus or virus-like structure makes contact with a precursor T-Helper cell or mature T-Helper cell by means of the virus's exterior probes or virus-like structure's exterior probes. Once the exterior probes engage the precursor target cells' or mature T-Helper cells' cell-surface receptors, the modified virus or virus-like structure inserts into the precursor T-Helper cells or mature T-Helper cells the modified genome it is carrying. As the precursor T-Helper cell matures it will either not express CXCR4 or CCR5 cell-surface receptors or it will express biologically nonfunctional CXCR4 or CCR5 cell surface receptors. Mature T-Helper cells with either remove their CXCR4 or CCR5 cell-surface receptors from the outer membrane or the existing CXCR4 or CCR5 cell-surface receptors will be made nonfunctional. By removing functional CXCR4 and CCR5 cell-surface receptors from the surface of mature T-Helper cells prevents HIV virions from being able to gain access to mature T-Helper cells and thus prevents AIDS.

### DETAILED DESCRIPTION

The medical treatment method described herein is a means of making the immune system impervious to infection by pathogens that utilize cell-surface receptors located on cells comprising the immune system to gain access to the cells that constitute the immune system. To make cells impervious to infection is a simple function of preventing such cells from ever expressing the cell-surface receptors that make them vulnerable to infection or alter the cell-surface receptors in a manner that prevents the cell-surface receptor from being used by a pathogen to gain access to the cell. It is known that the Human Immunodeficiency Virus utilizes its gp120 probe to engage the CD4 cell-surface receptor on its host the T-Helper cell. Once the gp120 probe has successfully engaged a CD4 cell-surface receptor, the HIV virion utilizes its gp41 probe to engage either a CXCR4 or CCR5 cell-surface receptor affixed to the surface of the T-Helper cell. Following successful engagement of a CXCR4 or CCR5 cell-surface receptor, the HIV virion is able to open a portal through the outer membrane of the T-Helper cell and insert into the T-Helper cell the HIV RNA genome and the enzymes needed to activate HIV's RNA genome. It is known that individuals that lack the gene to express a CCR5 cell-surface receptor on the surface of their T-Helper cells are immune to infection by the Human Immunodeficiency Virus.

T-Helper cells undergo a complex maturation process before they are released into circulation as mature T-Helper cells. Stem cells in the bone marrow produce precursor T-lymphocytes that leave the bone marrow and travel to the thymus gland. In the thymus gland these precursor T-lymphocytes or precursor T-Helper cells undergo various stages of development. As a precursor T-Helper cell develops into a mature T-Helper cell various cell-surface receptors are expressed on the surface of the precursor T-Helper cell. Some of the cell-surface receptors are expressed on the surface of the precursor T-Helper cell for a short time and then removed from the surface of the cell.

The medical treatment method described herein takes the concept that if a T-Helper cell does not express a cell-surface receptor that makes it vulnerable to infection by a pathogen then the cell is impervious to that pathogen and develops this concept into a practical treatment strategy to prevent infection by pathogens. Altering the genes that control the expression of the cell-surface receptors that make a cell vulnerable to infection prevents the infection. In the case of infection by the Human Immunodeficiency Virus altering the genes that control the expression of the CXCR4 and CCR5 cell-surface receptors prevents T-Helper cells from becoming infected by the Human Immunodeficiency Virus or other pathogens such as Yersinia pestis. The medical treatment method targets a precursor form of T-Helper cell as it develops but before it expresses either the CXCR4 or CCR5 cell-surface receptors on its outer membrane. Altering the genes that control the expression of the CXCR4 and CCR5 cell-surface receptors in a manner that prevents expression of these genes before the cell-surface receptors are generated dictates that the mature form of the T-Helper cell will not express either the CXCR4 cell-surface receptor or the CCR5 cell-surface receptor on its surface.

Viruses such as the Human Immunodeficiency Virus insert their genome into a cell, this genome gets transported from the cytoplasm to the nucleus of the cell, and then this genome gets inserted into the nuclear DNA of the cell. The Human Immunodeficiency Virus is designed to target T-Helper cells, and insert genetic material into T-Helper cells. The medical treatment method described herein is to take a modified form of a virus and use this modified virus to insert medically beneficial genetic material into specific cells to produce a medically beneficial response. In the case of preventing AIDS, the method is comprised of utilizing a modified virus to seek out and engage precursor T-Helper cells, whether this be stem cells or precursor T-Helper cells located in the bone marrow, in transit to the thymus gland or present in the thymus gland and insert into the precursor T-Helper cells genetic instruction code that alters the genes responsible for the CXCR4 and CCR5 cell-surface receptors. The modified virus would be generated with glycoprotein probes that would engage cell-surface receptors located on either stem cells or precursor T-Helper cells, that are unique to these cells, and offer the proper means for the modified virus to gain access to the precursor T-Helper cells so that the modified virus can deliver the medially therapeutic genome it carries to the precursor T-Helper cells. Cell-surface receptors such as CD7, CD 34, CD38, CD44 may designate precursor T-Helper cells. The presence of both the CD4 and CD8 cell-surface receptors designates a precursor form of T-Helper cell. Modified virus virions could use glycoprotein probes that seek out these cell-surface receptors in order to find and engage the proper precursor T-Helper cells.

STOP codes are biologic instructions that tell the polymerase molecules, that read and decipher the nuclear DNA, when to stop deciphering the code. Several forms of STOP codes have been defined. Utilizing the Universal Code the codons, comprised of three nucleic acids, designated TAA, TAG, and TGA represent STOP codes. Therefore, if a polymerase molecule which is reading a segment of nuclear DNA encounters a section that is comprised of three nucleic acids arranged as TAA, TAG, or TGA, the polymerase recognizes this particular instruction code as an instruction to cause the polymerase molecule to cease reading any further instruction code at that location in the nuclear DNA. Other biologic coding may interfere with the reading of the nuclear DNA and terminate deciphering of the nuclear DNA.

It is known that individuals that lack a 32-base segment in both CCR5 genes that they possess will not express a CCR5 cell-surface receptor on the surface of their mature T-Helper cells and such individuals are resistant to infection by the Human Immunodeficiency Virus. The medically therapeutic method described herein creates an alteration to CCR5 cell-surface receptor genes by inserting into the CCR5 cell-surface receptor genes a STOP code. This STOP code may occur anywhere in the gene to prevent the gene from being deciphered properly by a polymerase molecule. Mimicking mother nature, the STOP code could be inserted into the gene at the point along the gene's nucleic acid sequence that prevents the 32-base segment from being read in the gene similar to the naturally occurring gene mutation that results in individuals being resistant to the Yersinia pestis pathogen. STOP codes as seen in DNA are represented by any of the three codons: ‘TAA’, ‘TAG’, and ‘TGA’. STOP codes as seen in messenger RNA are represented by any of the three codons: ‘UAA’, ‘UAG’, and ‘UGA’.

The medical treatment method described herein is comprised of a virus that is modified in such a fashion that it seeks out precursor T-Helper cells, the virus further modified to carry medically therapeutic genetic instruction code and insert this medically therapeutic instruction code into the precursor T-Helper cell, with the intention that this medically therapeutic instruction code will alter the CXCR4 or CCR5 cell-surface gene in such a manner that the mature T-Helper cell will not express either a CXCR4 cell-surface receptor or a CCR5 cell-surface receptor on the surface of its outer membrane.

It is known that as cells develop toward maturation, immature precursor cells may express certain cell-surface receptors for a period of time and then either cause the cell-surface receptor to become nonfunctional or remove the cell-surface receptor from the surface of the cell. As precursor T-Helper cells proceed through a very complex maturation process to become mature T-Helper cells, certain cell-surface receptors are expressed on the surface of the precursor T-Helper cell and then later altered to be nonfunctional or removed from the surface of the precursor T-Helper cell. Given that there is a natural process already in existence to cause an existing cell-surface receptor affixed to the outside membrane of a cell to be altered to become nonfunctional or to be physically removed from the surface of the cell, this feature can be utilized to remove unwanted cell-surface receptors from existing cells to produce a beneficial medically therapeutic outcome. In a patient infected with Human Immunodeficiency Virus, the patient's healthy, noninfected T-Helper cells are vulnerable to infection by the Human Immunodeficiency Virus by the fact the noninfected cells express CXCR4 or CCR5 cell-surface receptors on their surface. Modifying the Human Immunodeficiency Virus to carry a biologic instruction code to cause a healthy noninfected T-Helper cell to make the CXCR4 and CCR5 cell-surface receptors it expresses on its surface to either become nonfunctional or remove these cell-surface receptors from the surface of its outer membrane will result in noninfected T-Helper cells being impervious to infection by the Human Immunodeficiency Virus. Further, modifying the Human Immunodeficiency Virus to carry a biologic instruction code to cause infected T-Helper cells to make the FasL cell-surface receptors it carries to become nonfunctional or cause the FasL cell-surface receptors to be removed from the surface will prevent infected T-Helper cells from triggering apoptosis in noninfected T-Helper cells, which will prevent AIDS from occurring.

Viruses or virus-like structures can be fashioned to act as transport vehicles to carry and deliver medically therapeutic genetic material, representing a quantity of either RNA segments or DNA segments, directly to specific precursor T-Helper cells or mature T-Helper cells.

Naturally occurring viruses can be altered by replacing the genetic material the virus would carry, with medically therapeutic RNA genome molecules that would have a medically beneficial therapeutic effect on precursor T-Helper cells and on mature T-Helper cells.

Naturally occurring viruses can be further modified to have their naturally occurring glycoprotein surface probes replaced by glycoprotein surface probes that target specific cells in the body. Viruses modified to carry and deliver medically therapeutic RNA genome molecules as the payload, further modified to have their glycoprotein surface probes, that cause the modified virus to engage specific precursor T-Helper cells or mature T-Helper cells in the body, provides a method whereby specific cells in the body can be targeted and cell-surface receptors that appear or would appear on the surface of mature T-Helper cells are altered to produce a medically therapeutic result.

Virus-like structures can be constructed with similar physical characteristics to naturally occurring viruses and be fashioned to carry medically therapeutic RNA molecules as their payload and have located on the surface glycoprotein probes that engage precursor T-Helper cells or mature T-Helper cells in the body. Viruses-like structures carrying medically therapeutic drug molecules as their payload, constructed to have their glycoprotein surface probes engage specific cells in the body, and deliver to those specific cells the drug the virus-like structures carry provides a device whereby specific cells in the body can be targeted and this device embodies a means of providing to a specific type of cell in the body a drug to participate in chemical reactions with the intent to accomplish a medically therapeutic outcome. The advantage of a virus-like structure is that the physical dimensions of the virus-like structure can be adjusted to accommodate variations in the physical size of the payload, yet maintain a means of engaging targeted cells in the body and delivering to those targeted cells the medically therapeutic RNA genome molecules required to accomplish the desired medical therapeutic outcome. A second advantage of utilizing virus-like structures is to be able to change the surface characteristics of the transport vehicle to prevent the body's immune system from reacting to the presence of the therapeutic modified virus and destroying the modified virus before it is able to deliver the payload it carries to the cells it has been designed to target. HIV utilizes an exterior envelope comprised of the surface membrane of its host, the T-Helper cell, which acts as a disguise to fool the body's immune system detection resources. Virus-like structures could be fashioned, similar to HIV, to have as an exterior envelope a surface that resembles a cell's outer membrane in order to avoid detection by the body's immune system to improve survivability and functionality of the virus-like structure.

Replicating viruses and constructing viruses to carry DNA payloads is a form of manufacturing technology that has already been well established and is in use facilitating the concept of gene therapy. Replicating viruses and designing these viruses to carry drug as the genetic payload would incorporate similar techniques as already proven useful in current DNA gene therapy technologies.

To carry out the process to manufacture a modified medically therapeutic Human Immunodeficiency Virus virions, DNA or RNA that would code for the general physical structures of the Human Immunodeficiency Virus virion would be inserted into a host. The host may include devices such as a host cell or a hybrid host cell. The host may utilize DNA or RNA or a combination of genetic instructions in order to accomplish the construction of medically therapeutic virus virions. The DNA or messenger RNA molecules to create the medically therapeutic virus virions would direct the cells to generate copies of the medically therapeutic Human Immunodeficiency Virus virions carrying a medically therapeutic drug payload. In some cases DNA or messenger RNA would be inserted into the host that would be coded to cause the production of surface probes that would be affixed to the surface of the Human Immunodeficiency Virus virions that would target the surface receptors on specific cells in the body other than the T-Helper cells the Human Immunodeficiency Virus virions naturally targets. DNA or messenger RNA would direct the host to generate copies of the medically therapeutic RNA genome molecules that would provide a therapeutic action; these medically therapeutic RNA genome molecules would take the place of the Human Immunodeficiency Virus's innate genome as its payload. The medical treatment form of the Human Immunodeficiency Virus carrying medically therapeutic RNA genome molecules would be produced, assembled and released from a host. Virus-like structures would be generated in similar fashion using a host such as host-cells or hybrid host cells. The copies of the medically therapeutic Human Immunodeficiency Virus virions or virus-like structures, upon exiting the host, would be collected, stored and utilized as a medical treatment as necessary.

A method of preventing and treating AIDS could be to take the naturally occurring Human Immunodeficiency Virus (HIV) which is an RNA virus and replace HIV's innate genome with a quantity of medially therapeutic RNA molecules. Utilizing modified virus virions to insert medically therapeutic RNA genome molecules into precursor T-Helper cells or mature T-Helper cells, the instruction code carried in the RNA genome that will alter cell-surface genes in the precursor T-Helper cells or cause existing cell-surface receptors to become nonfunctional in mature T-Helper cells would prevent HIV from being able to infect a patient's T-Helper cell population and thus stop AIDS from occurring or progressing in a patient.

The modified Human Immunodeficiency Virus virions and virus-like structures would be incapable of replication on their own due to the fact that the messenger RNA that would code for the replication process to produce copies of the virus or virus-like structure would not be present in the modified form of the Human Immunodeficiency Virus virions or virus-like structure.

The described medical method includes a quantity of modified Human Immunodeficiency Virus virions or modified viruses or virus-like structures introduced into a patient's blood stream or tissues so that the modified virus or virus-like structure could deliver the medially therapeutic RNA genome payload that it carries to precursor T-Helper cells or mature T-Helper cells in the body. By utilizing the described method to provide the precursor T-Helper cells of the body with a modified genome to create an altered CXCR4 and CCR5 cell-surface receptors to be expressed on mature T-Helper cells prevents infection by HIV and prevents progression to AIDS.

### DRAWING

None.

The terms and expressions which are employed here are used as terms of description and are not limitation and there is no intention, in the use of terms and expressions, of excluding equivalents of the features presented, and described, or portions thereof, it being recognized that various modifications are possible in the scope of the invention or process as claimed.

What is claimed:

1. A medical treatment method for inserting a quantity of medically therapeutic genetic material into stem cells in the body comprising:
(a) a quantity of modified virus virions fashioned for the purpose of transporting a quantity of said medically therapeutic genetic material,
(b) said quantity of said modified virus virions having a quantity of glycoprotein probes affixed to their surface, said glycoprotein probes constructed in a manner to target said stem cells in said body,
(c) said glycoprotein probes capable of engaging specific cell-surface receptors on said stem cells,
(d) once said glycoprotein probes have successfully engaged cell-surface receptors on said stem cells, said quantity of said modified virus virions deliver into said stem cells a quantity of said medically therapeutic genetic material said modified virus virions are carrying,
(e) once said glycoprotein probes have successfully engaged cell-surface receptors on said stem cells, said quantity of said modified virus virions deliver into said stem cells a quantity of enzymes to be utilized to transcribe said quantity of said medically therapeutic genetic material said for the purpose of converting said medically therapeutic genetic material into functional deoxyribonucleic acid molecules,
(f) once said glycoprotein probes have successfully engaged cell-surface receptors on said stem cells, said quantity of said modified virus virions deliver into said stem cells a quantity of enzymes to be utilized to modify the medically therapeutic genetic material or resultant medically therapeutic deoxyribonucleic acid molecules to a functional form that can be inserted into said stem cells' own native nuclear deoxyribonucleic acid genome,
(g) once said glycoprotein probes have successfully engaged cell-surface receptors on said stem cells, said quantity of said modified virus virions deliver into said stem cells a quantity of enzymes to be utilized to transport the functional form of said modified medically therapeutic deoxyribonucleic acid molecules and properly integrate said modified medically therapeutic deoxyribonucleic acid molecules into said stem cells' own native nuclear deoxyribonucleic acid genome,
(h) said medically therapeutic genetic material converted into medically therapeutic functional deoxyribonucleic acid molecules inserted into said stem cells' own native deoxyribonucleic acid genome, are to cause an alteration to said stem cells' cell-surface receptor genes to eventually cause descendents of said stem cells, the mature form of T-Helper cells, not to express a quantity of cell-surface receptors on their surface for the purpose of preventing any pathogen that would utilize said cell-surface receptors from engaging said cell-surface receptors, thus preventing said pathogens from gaining access to said mature T-Helper cells,
(i) said medically therapeutic genetic material converted into medically therapeutic functional deoxyribonucleic acid molecules inserted into said stem cells' own native deoxyribonucleic acid genome, are to cause an alteration to said stem cells' cell-surface receptor genes to eventually cause descendents of said stem cells, the mature form of T-Helper cells, to express a quantity of altered nonfunctional cell-surface receptors for the purpose of preventing any pathogen that would utilize such said cell-surface receptors from engaging said cell-surface receptors, thus preventing said pathogens from gaining access to said mature T-Helper cells,
whereby altering the genes responsible for specific cell-surface receptors utilized by pathogens to gain access to said mature form of T-Helper cells, said mature T-Helper cells are resistant to infection by said pathogens and thus avert the disease that would be potentially caused by said pathogens.

2. The medical treatment method in claim 1 wherein said medically therapeutic genetic material is a quantity of ribonucleic acid molecules.

3. The medical treatment method in claim 1 wherein said modified virus virions selected from the group consisting of naturally occurring virus virions whose payload has been altered to carry a quantity of said medically therapeutic genetic material, naturally occurring virus virions whose payload has been altered to carry a quantity of said medically therapeutic genetic material said virus virions having their surface glycoprotein probes altered in a manner the glycoprotein probes are fashioned to engage said stem cells in said body, and virus-like structures constructed to resemble naturally occurring virus virions said virus-like structures capable of carrying a quantity of said medically therapeutic genetic material said virus-like structures constructed with glycoprotein probes fashioned to engage said stem cells in said body said virus-like structures capable of delivering said quantity of medically therapeutic genetic material to said stem cells in said body.

4. The medical treatment method in claim 1 wherein said body is comprised of the physical features of the human body.

5. The medical treatment method in claim 1 wherein said medically therapeutic genetic material is a quantity of ribonucleic acid molecules comprised of biologic instruction code intended to cause a mutation to the genetic instruction code of the CXCR4 gene present in the native deoxyribonucleic acid of said stem cells, whereby said medically therapeutic ribonucleic acid molecules are coded with a quantity of biologic instructions such that descendents of said stem cells, said mature T-Helper cells, where said alteration to said CXCR4 cell-surface receptor gene will cause said mature T-Helper cells not express any form of said CXCR4 cell-surface receptor on their surface, or said alteration to said CXCR4 cell-surface receptor gene will cause said mature T-Helper cell to express biologically nonfunctional CXCR4 cell-surface receptors, and
whereby preventing said CXCR4 cell-surface receptors from being expressed on the surface of said mature T-Helper cells, or altering said CXCR4 cell-surface receptors to make them biologically nonfunctional said mature T-Helper cells will be made impervious to infection by any pathogen, including the Human Immunodeficiency Virus, which utilizes said CXCR4 cell-surface receptors to gain access to said mature T-Helper cells.

6. The medical treatment method in claim 1 wherein said medically therapeutic genetic material is a quantity of ribonucleic acid molecules comprised of biologic instruction code intended to cause a mutation to the genetic instruction code of the CCR5 gene present in the native deoxyribonucleic acid of said stem cells, whereby said medically therapeutic ribonucleic acid molecules are coded with a quantity of biologic instructions such that descendents of said stem cells, said mature T-Helper cells, where said alteration to said CCR5 cell-surface receptor gene will cause said mature T-Helper cells not express any form of said CCR5 cell-surface receptor on their surface, or said alteration to said CCR5 cell-surface receptor gene will cause said mature T-Helper cell to express biologically nonfunctional CCR5 cell-surface receptors, and
whereby preventing said CCR5 cell-surface receptors from being expressed on the surface of said mature T-Helper cells, or altering said CCR5 cell-surface receptors to make them biologically nonfunctional said mature T-Helper cells will be made impervious to infection by any pathogen, including the Human Immunodeficiency Virus, which utilizes said CCR5 cell-surface receptors to gain access to said mature T-Helper cells.

7. The medical treatment method in claim 1 wherein said medically therapeutic genetic material is a quantity ribonucleic acid molecules, which is genetic instruction codes that when transcribed into deoxyribonucleic acid represent a quantity of STOP codes,
whereby when said quantity of STOP codes are inserted into a gene in a chromosome this will result in a medically therapeutic benefit,
whereby when said quantity of STOP codes are inserted into a gene in a chromosome, said quantity of STOP codes cause the polymerase molecules that read and decipher said deoxyribonucleic acid to prematurely cease reading and deciphering the instruction code present in said gene,
whereby said quantity of STOP codes when inserted into said gene that is responsible for the construction of said CXCR4 cell-surface receptors will cause either said mature T-Helper cells not to express said CXCR4 cell-surface receptors on the surface of said mature T-Helper cells or cause biologically nonfunctional CXCR4 cell-surface receptors to be expressed on the surface of said mature T-Helper cells due to inaccurate deciphering of the instruction code present in said gene,
whereby said quantity of STOP codes when inserted into said gene that is responsible for the construction of said CCR5 cell-surface receptors will cause either said mature T-Helper cells not to express said CCR5 cell-surface receptors on the surface of said mature T-Helper cells or cause biologically nonfunctional CCR5 cell-surface receptors to be expressed on the surface of said mature T-Helper cells due to inaccurate deciphering of the instruction code present in said gene, and
whereby the action of inserting a quantity of said STOP codes into said genes responsible for the proper construction and positioning of cell-surface receptors makes said mature T-Helper cells impervious to pathogens that would use said cell-surface receptors to gain access to said mature T-Helper cells.

8. The medical treatment method in claim 1 wherein said medically therapeutic genetic material is a quantity of ribonucleic acid molecules which is a quantity of STOP codes selected from the group consisting of ‘UAA’, ‘UAG’ and ‘UGA’.

9. The medical treatment method in claim 1 wherein said modified virus virions are a modified form of Human Immunodeficiency Virus virions,
whereby the native ribonucleic acid genome said Human Immunodeficiency Virus virions naturally carry has been altered to in a manner to produce a medically therapeutic benefit,
whereby said enzymes said modified Human Immunodeficiency Virus virions carry are modified in a fashion to successfully assist said medically therapeutic genetic material said modified Human Immunodeficiency Virus virions carry to be properly inserted into said stem cells' nuclear deoxyribonucleic acid to achieve the effect required to properly alter said cell-surface receptors that appear on said mature T-Helper cells to make said mature T-Helper cells impervious to infection by pathogens, and
whereby the glycoprotein probes that appear on the surface of the naturally occurring Human Immunodeficiency Virus virions have been modified such that said Human Immunodeficiency Virus virions target said stem cells as the host cells they actively intend to engage and deliver to said stem cells said payload of said medically therapeutic genetic material and said enzymes said modified Human Immunodeficiency Virus virions carry.

10. The medical treatment method in claim 1 wherein said enzyme molecules include a quantity of reverse transcriptases, a quantity of integrases, and a quantity of proteases,
whereby said quantity of reverse transcriptases change ribonucleic acid to deoxyribonucleic acid inside said stem cells,
whereby said quantity of proteases cleave said medically therapeutic genetic material or its products at specific places along the chain of nucleic acids to produce the biologically functional form of said deoxyribonucleic acid to be inserted into said stem cells' nuclear deoxyribonucleic acid,
whereby the functional form of said medically therapeutic genetic material which have been transcribed into functional deoxyribonucleic acid molecules represents the instruction code to be inserted said stem cells' nuclear deoxyribonucleic acid,
whereby the functional form of said medically therapeutic genetic material inserted into the nuclear deoxyribonucleic acid of said stem cells will cause alteration to cell-surface receptors to achieve a medically therapeutic benefit,
whereby said quantity of integrase enzymes transport the functional form of said medically therapeutic genetic material which has been transcribed into deoxyribonucleic acid and inserts this into the nuclear deoxyribonucleic acid of said stem cells in a position in the stem cells' nuclear deoxyribonucleic acid that results in a medically therapeutic benefit, and
whereby said quantity of integrase enzymes transport the functional form of said medically therapeutic genetic material which has been transcribed into deoxyribonucleic acid and inserts this into the nuclear deoxyribonucleic acid of said stem cells in a position in the stem cells' nuclear deoxyribonucleic acid that results in cell-surface receptor genes present in the deoxyribonucleic acid being altered such that certain cell-surface receptors are either not expressed on the surface of said mature T-Helper cells or certain cell-surface receptors are expressed as nonfunctional cell-surface receptors on the surface of mature T-Helper cells.

11. A medical treatment method for inserting a quantity of medically therapeutic genetic material into precursor T-Helper cells in the body comprising:
(a) a quantity of modified virus virions fashioned for the purpose of transporting a quantity of said medically therapeutic genetic material,
(b) said quantity of said modified virus virions having a quantity of glycoprotein probes affixed to their surface, said glycoprotein probes constructed in a manner to target said precursor T-Helper cells in said body,
(c) said glycoprotein probes capable of engaging specific cell-surface receptors on said precursor T-Helper cells,
(d) once said glycoprotein probes have successfully engaged cell-surface receptors on said precursor T-Helper cells, said quantity of said modified virus virions deliver into said precursor T-Helper cells a quantity of said medically therapeutic genetic material said modified virus virions are carrying,
(e) once said glycoprotein probes have successfully engaged cell-surface receptors on said precursor T-Helper cells, said quantity of said modified virus virions deliver into said precursor T-Helper cells a quantity of enzymes to be utilized to transcribe said quantity of said medically therapeutic genetic material said for the purpose of converting said medically therapeutic genetic material into functional deoxyribonucleic acid molecules,
(f) once said glycoprotein probes have successfully engaged cell-surface receptors on said precursor T-Helper cells, said quantity of said modified virus virions deliver into said precursor T-Helper cells a quantity of enzymes to be utilized to modify the medically therapeutic genetic material or resultant medically therapeutic deoxyribonucleic acid molecules to a functional form that can be inserted into said precursor T-Helper cells' own native nuclear deoxyribonucleic acid genome,
(g) once said glycoprotein probes have successfully engaged cell-surface receptors on said precursor T-Helper cells, said quantity of said modified virus virions deliver into said precursor T-Helper cells a quantity of enzymes to be utilized to transport the functional form of said modified medically therapeutic deoxyribonucleic acid molecules and properly integrate said modified medically therapeutic deoxyribonucleic acid molecules into said precursor T-Helper cells' own native nuclear deoxyribonucleic acid genome,
(h) said medically therapeutic genetic material converted into medically therapeutic functional deoxyribonucleic acid molecules inserted into said precursor T-Helper cells' own native deoxyribonucleic acid genome, are to cause an alteration to said precursor T-Helper cells' cell-surface receptor genes to eventually cause descendents of said precursor T-Helper cells, the mature form of T-Helper cells, not to express a quantity of cell-surface receptors on their surface for the purpose of preventing any pathogen that would utilize said cell-surface receptors from engaging said cell-surface receptors, thus preventing said pathogens from gaining access to said mature T-Helper cells,
(i) said medically therapeutic genetic material converted into medically therapeutic functional deoxyribonucleic acid molecules inserted into said precursor T-Helper cells' own native deoxyribonucleic acid genome, are to cause an alteration to said precursor T-Helper cells' cell-surface receptor genes to eventually cause descendents of said precursor T-Helper cells, the mature form of T-Helper cells, to express a quantity of altered nonfunctional cell-surface receptors for the purpose of preventing any pathogen that would utilize such said cell-surface receptors from engaging said cell-surface receptors, thus preventing said pathogens from gaining access to said mature T-Helper cells,
whereby altering the genes responsible for specific cell-surface receptors utilized by said pathogens to gain access to said mature form of T-Helper cells, said mature T-Helper cells are resistant to infection by said pathogens and thus avert the disease that would be potentially caused by said pathogens.

12. The medical treatment method in claim 11 wherein said medically therapeutic genetic material is a quantity of ribonucleic acid molecules.

13. The medical treatment method in claim 11 wherein said modified virus virions selected from the group consisting of naturally occurring virus virions whose payload has been altered to carry a quantity of said medically therapeutic genetic material, naturally occurring virus virions whose payload has been altered to carry a quantity of said medically therapeutic genetic material said virus virions having their surface glycoprotein probes altered in a manner the glycoprotein probes are fashioned to engage said precursor T-Helper cells in said body, and virus-like structures constructed to resemble naturally occurring virus virions said virus-like structures capable of carrying a quantity of said medically therapeutic genetic material said virus-like structures constructed with glycoprotein probes fashioned to engage said precursor T-Helper cells in said body said virus-like structures capable of delivering said quantity of medically therapeutic genetic material to said precursor T-Helper cells in said body.

14. The medical treatment method in claim 11 wherein said body is comprised of the physical features of the human body.

15. The medical treatment method in claim 11 wherein said medically therapeutic genetic material is a quantity of ribonucleic acid molecules comprised of biologic instruction code intended to cause a mutation to the genetic instruction code of the CXCR4 gene present in the native deoxyribonucleic acid of said precursor T-Helper cells,
whereby said medically therapeutic ribonucleic acid molecules are coded with a quantity of biologic instructions such that descendents of said precursor T-Helper cells, said mature T-Helper cells, where said alteration to said CXCR4 cell-surface receptor gene will cause said mature T-Helper cells not express any form of said CXCR4 cell-surface receptor on their surface, or said alteration to said CXCR4 cell-surface receptor gene will cause said mature T-Helper cell to express biologically nonfunctional CXCR4 cell-surface receptors, and
whereby preventing said CXCR4 cell-surface receptors from being expressed on the surface of said mature T-Helper cells, or altering said CXCR4 cell-surface receptors to make them biologically nonfunctional said mature T-Helper cells will be made impervious to infection by any pathogen, including the Human Immunodeficiency Virus, which utilizes said CXCR4 cell-surface receptors to gain access to said mature T-Helper cells.

16. The medical treatment method in claim 11 wherein said medically therapeutic genetic material is a quantity of ribonucleic acid molecules comprised of biologic instruction code intended to cause a mutation to the genetic instruction code of the CCR5 gene present in the native deoxyribonucleic acid of said precursor T-Helper cells,
whereby said medically therapeutic ribonucleic acid molecules are coded with a quantity of biologic instructions such that descendents of said precursor T-Helper cells, said mature T-Helper cells, where said alteration to said CCR5 cell-surface receptor gene will cause said mature T-Helper cells not express any form of said CCR5 cell-surface receptor on their surface, or said alteration to said CCR5 cell-surface receptor gene will cause said mature T-Helper cell to express biologically nonfunctional CCR5 cell-surface receptors, and
whereby preventing said CCR5 cell-surface receptors from being expressed on the surface of said mature T-Helper cells, or altering said CCR5 cell-surface receptors to make them biologically nonfunctional said mature T-Helper cells will be made impervious to infection by any pathogen, including the Human Immunodeficiency Virus, which utilizes said CCR5 cell-surface receptors to gain access to said mature T-Helper cells.

17. The medical treatment method in claim 11 wherein said medically therapeutic genetic material is a quantity ribonucleic acid molecules, which is genetic instruction codes that when transcribed into deoxyribonucleic acid represent a quantity of STOP codes,
whereby when said quantity of STOP codes are inserted into a gene in a chromosome this will result in a medically therapeutic benefit,
whereby when said quantity of STOP codes are inserted into a gene in a chromosome, said quantity of STOP codes cause the polymerase molecules that read and decipher said deoxyribonucleic acid to prematurely cease reading and deciphering the instruction code present in said gene,
whereby said quantity of STOP codes when inserted into said gene that is responsible for the construction of said CXCR4 cell-surface receptors will cause either said mature T-Helper cells not to express said CXCR4 cell-surface receptors on the surface of said mature T-Helper cells or cause biologically nonfunctional CXCR4 cell-surface receptors to be expressed on the surface of said mature T-Helper cells due to inaccurate deciphering of the instruction code present in said gene,
whereby said quantity of STOP codes when inserted into said gene that is responsible for the construction of said CCR5 cell-surface receptors will cause either said mature T-Helper cells not to express said CCR5 cell-surface receptors on the surface of said mature T-Helper cells or cause biologically nonfunctional CCR5 cell-surface receptors to be expressed on the surface of said mature T-Helper cells due to inaccurate deciphering of the instruction code present in said gene, and
whereby the action of inserting a quantity of said STOP codes into said genes responsible for the proper construction and positioning of cell-surface receptors makes said mature T-Helper cells impervious to pathogens that would use said cell-surface receptors to gain access to said mature T-Helper cells.

18. The medical treatment method in claim 11 wherein said medically therapeutic genetic material is a quantity of ribonucleic acid molecules which is a quantity of STOP codes selected from the group consisting of ‘UAA’, ‘UAG’ and ‘UGA’.

19. The medical treatment method in claim 11 wherein said modified virus virions are a modified form of Human Immunodeficiency Virus virions,
whereby the native ribonucleic acid genome said Human Immunodeficiency Virus virions naturally carry has been altered to in a manner to produce a medically therapeutic benefit,
whereby said enzymes said modified Human Immunodeficiency Virus virions carry are modified in a fashion to successfully assist said medically therapeutic genetic material said modified Human Immunodeficiency Virus virions carry to be properly inserted into said precursor T-Helper cells' nuclear deoxyribonucleic acid to achieve the effect required to properly alter said cell-surface receptors that appear on said mature T-Helper cells to make said mature T-Helper cells impervious to infection by pathogens, and
whereby the glycoprotein probes that appear on the surface of the naturally occurring Human Immunodeficiency Virus virions have been modified such that said Human Immunodeficiency Virus virions target said precursor T-Helper cells as the host cells they actively intend to engage and deliver to said precursor T-Helper cells said payload of said medically therapeutic genetic material and said enzymes said modified Human Immunodeficiency Virus virions carry.

20. The medical treatment method in claim 11 wherein said enzyme molecules include a quantity of reverse transcriptases, a quantity of integrases, and a quantity of proteases,
whereby said quantity of reverse transcriptases change ribonucleic acid to deoxyribonucleic acid inside said precursor T-Helper cells,
whereby said quantity of proteases cleave said medically therapeutic genetic material or its products at specific places along the chain of nucleic acids to produce the biologically functional form of said deoxyribonucleic acid to be inserted into said precursor T-Helper cells' nuclear deoxyribonucleic acid,
whereby the functional form of said medically therapeutic genetic material which have been transcribed into functional deoxyribonucleic acid molecules represents the instruction code to be inserted said precursor T-Helper cells' nuclear deoxyribonucleic acid,
whereby the functional form of said medically therapeutic genetic material inserted into the nuclear deoxyribonucleic acid of said precursor T-Helper cells will cause alteration to cell-surface receptors to achieve a medically therapeutic benefit,
whereby said quantity of integrase enzymes transport the functional form of said medically therapeutic genetic material which has been transcribed into deoxyribonucleic acid and inserts this into the nuclear deoxyribonucleic acid of said precursor T-Helper cells in a position in the precursor T-Helper cells' nuclear deoxyribonucleic acid that results in a medically therapeutic benefit, and
whereby said quantity of integrase enzymes transport the functional form of said medically therapeutic genetic material which has been transcribed into deoxyribonucleic acid and inserts this into the nuclear deoxyribonucleic acid of said precursor T-Helper cells in a position in the precursor T-Helper cells' nuclear deoxyribonucleic acid that results in cell-surface receptor genes present in the deoxyribonucleic acid being altered such that certain cell-surface receptors are either not expressed on the surface of said mature T-Helper cells or certain cell-surface receptors are expressed as nonfunctional cell-surface receptors on the surface of mature T-Helper cells.

21. The medical treatment method in claim 11 wherein said glycoprotein probes include glycoprotein probes that are capable of engaging the Cluster Designation 4 cell-surface receptors and glycoprotein probes engaging the Cluster Designation 8 cell-surface receptors affixed to the surface of precursor T-Helper cells,
whereby the modified virus virions utilize the Cluster Designation 4 and Cluster Designation 8 cell-surface receptors to gain access to said precursor T-Helper cells and deliver to said precursor T-Helper cells said genetic material and said enzymes they carry.

22. The medical treatment method in claim 11 wherein said glycoprotein probes that are capable of engaging cell-surface receptors selected from the group consisting of Cluster Designation 4 cell-surface receptors, Cluster Designation 7 cell-surface receptors, Cluster Designation 8 cell-surface receptors, Cluster Designation 25 cell-surface receptors, Cluster Designation 34 cell-surface receptors, Cluster Designation 38 cell-surface receptors, and Cluster Designation 44 cell-surface receptors,
whereby the modified virus virions utilize the Cluster Designation cell-surface receptors to gain access to said precursor T-Helper cells and deliver to said precursor T-Helper cells said genetic material and said enzymes they carry.

23. A medical treatment method for inserting a quantity of medically therapeutic genetic material into T-Helper cells in the body comprising:
(a) a quantity of modified virus virions fashioned for the purpose of transporting a quantity of said medically therapeutic genetic material,
(b) said modified virus virions carrying enzymes to properly implement said medically therapeutic genetic material once said medically therapeutic genetic material have been inserted into said T-Helper cells,
(c) said modified virus virions having a quantity of glycoprotein probes affixed to their surface, said glycoprotein probes constructed in a manner to target said T-Helper cells inside said body,
(d) said glycoprotein probes capable of engaging specific cell-surface receptors affixed to the surface said T-Helper cells,
(e) once said glycoprotein probes located on said modified virus virions have successfully engaged said specific cell-surface receptors on said T-Helper cells, said modified virus virions deliver into said T-Helper cells a quantity of said medically therapeutic genetic material said modified virus virions are carrying,
(f) once said glycoprotein probes located on said modified virus virions have successfully engaged said specific cell-surface receptors on said T-Helper cells, said modified virus virions deliver into said T-Helper cells a quantity of said enzyme molecules said modified virus virions are carrying,
whereby said medically therapeutic genetic material will cause an alteration to the functionality of a quantity of cell-surface receptors to be affixed to the surface of said T-Helper cells with the result being to treat a medical disease.

24. The medical treatment method in claim 23 wherein said medically therapeutic genetic material is a quantity of ribonucleic acid molecules.

25. The medical treatment method in claim 23 wherein said modified virus virions are a modified form of Human Immunodeficiency Virus virions,
whereby the ribonucleic acid genome has been altered to in a manner to produce a medically therapeutic benefit,
whereby the enzymes said modified Human Immunodeficiency Virus virions carry are modified in a fashion to successfully assist the medically therapeutic genetic material said modified Human Immunodeficiency Virus virion carries to be properly inserted into said T-Helper cells' nuclear deoxyribonucleic acid to achieve the effect required to properly alter said cell-surface receptors that appear on said T-Helper cells to make said T-Helper cells impervious to infection by pathogens.

26. The medical treatment method in claim 23 wherein said medically therapeutic genetic material is a quantity of ribonucleic acid molecules comprised of biologic instruction code intended to cause said T-Helper cells to remove CXCR4 cell-surface receptors from their surface or make existing CXCR4 cell-surface receptors biologically nonfunctional,
whereby said medically therapeutic genetic material is coded with a quantity of biologic instructions that will cause said T-Helper cells to remove from their surface any form of CXCR4 cell-surface receptors or cause said T-Helper cells to alter existing CXCR4 cell-surface receptors to a form where said CXCR4 cell-surface receptors are biologically nonfunctional, unable to be successfully engaged by any glycoprotein probe on the surface of a Human Immunodeficiency Virus virion.

27. The medical treatment method in claim 23 wherein said medically therapeutic genetic material is a quantity of ribonucleic acid molecules comprised of biologic instruction code intended to cause said T-Helper cells to remove CCR5 cell-surface receptors from their surface or make existing CCR5 cell-surface receptors biologically nonfunctional,
whereby said medically therapeutic genetic material are coded with a quantity of biologic instructions that will cause said T-Helper cells to remove from their surface any form of said CCR5 cell-surface receptors or cause said T-Helper cells to alter existing said CCR5 cell-surface receptors to a form where said CCR5 cell-surface receptors are biologically nonfunctional, unable to be successfully engaged by any glycoprotein probe on the surface of a Human Immunodeficiency Virus virion.

28. The medical treatment method in claim 23 wherein said medically therapeutic genetic material is a quantity of ribonucleic acid molecules comprised of biologic instruction code intended to cause T-Helper cells infected with Human Immunodeficiency virus genome to remove FasL cell-surface receptors from their surface or make existing FasL cell-surface receptors biologically nonfunctional,
whereby said medically therapeutic genetic material are coded with a quantity of biologic instructions that will cause said T-Helper cells infected with the Human Immunodeficiency Virus genome to remove from their surface any form of FasL cell-surface receptors or cause said T-Helper cells infected with Human Immunodeficiency Virus genome to alter existing FasL cell-surface receptors to a form where said FasL cell-surface receptors are biologically nonfunctional, such that said FasL cell-surface receptors are unable to successfully engage Fas cell-surface receptors affixed to the surface of healthy noninfected T-Helper cells, thus T-Helper cells infected with the Human Immunodeficiency Virus genome are unable to terminate healthy noninfected T-Helper cells.

29. The medical treatment method in claim 23 wherein said enzyme molecules include a quantity of reverse transcriptases, a quantity of integrases, and a quantity of proteases,
whereby said quantity of reverse transcriptases change ribonucleic acid to deoxyribonucleic acid inside said T-Helper cells,
whereby said quantity of proteases cleave said medically therapeutic genetic material or its products at specific places along the chain of nucleic acids to produce the functional portion of said medically therapeutic genetic material,
whereby the functional portion of said medically therapeutic genetic material instruction code to be inserted into said T-Helper cells' nuclear deoxyribonucleic acid,
whereby the functional portion of said medically therapeutic genetic code transcribed into deoxyribonucleic acid inserted into the nuclear deoxyribonucleic acid of said T-Helper cell will cause alteration to cell-surface receptors to a achieve a medically therapeutic benefit, and
whereby said quantity of integrase enzymes transport the functional portion of said medically therapeutic genetic material which has been transcribed into deoxyribonucleic acid and inserts this into the deoxyribonucleic acid of said T-Helper cells in a position in the nuclear deoxyribonucleic acid that results in a medically therapeutic benefit.
